(19) United States
(12) Patent Application Publication
(10) Pub. No.: US 2024/0068046 A1
(43) Pub. Date: Feb. 29, 2024

(54) METHODS OF ANALYSIS OF ALLELIC EXPRESSION OF PIK3CA IN CANCER AND USES THEREOF

(71) Applicant: UNIVERSIDADE DO ALGARVE, Faro (PT)

(72) Inventors: Ana Teresa LUÍS LOPES MAIA, Faro (PT); Joana GONÇALVES DE GOUVEIA MAIA XAVIER, Faro (PT); Lizelle WINKELSTRÖTER CORREIA, Mafra (PT); Ramiro Emanuel MAGNO MORGADO, Faro (PT)

(73) Assignee: UNIVERSIDADE DO ALGARVE, Faro (PT)

(21) Appl. No.: 18/460,549

(22) Filed: Sep. 3, 2023

Related U.S. Application Data
(63) Continuation-in-part of application No. PCT/EP2022/055637, filed on Mar. 4, 2022.

(30) Foreign Application Priority Data
Mar. 5, 2021 (EP) 21161118.1
Mar. 5, 2021 (PT) 20211000008758

(51) Int. Cl. C12Q 1/6886

(52) U.S. Cl. CPC C12Q 1/6886; C12Q 2600/158; C12Q 2600/156; C12Q 2600/118; C12Q 2600/106

(57) ABSTRACT

The invention provides a method of identifying a subset of cancer patients with a poor prognosis, comprising comparing the allelic expression of a mutant and wildtype PIK3CA allele in a patient sample. The invention further teaches the use of drugs targeting the mutant PI3K protein in a subset of patients which are determined to express the a mutant PIK3CA allele above a certain threshold, in addition to a kit, or system with which PIK3CA allelic expression may be assessed.

### CROSS REFERENCE TO RELATED APPLICATIONS

This is a Continuation-in-Part of International Patent Application No. PCT/EP2022/055637 filed on Mar. 4, 2022; which claims priority to Portuguese National Patent Application No. 20211000008758, filed on Mar. 5, 2021, and European Patent Application No. 21161118.1, filed on Mar. 5, 2021, and both of which are incorporated by reference herein.

### FIELD

This invention provides a method of determining the allelic expression of the oncogene PIK3CA as a biomarker for breast cancer outcomes, or to identify a subset of patients with a likelihood of responsiveness to PI3K inhibitor medication.

### BACKGROUND

Humans are diploid organisms, and it possible that two alleles of a genetic locus in a heterozygous individual are expressed at a different rate, termed allelic imbalance. Allelic imbalance in gene expression can be generated by cis-regulatory variants, single base changes that modulate gene expression levels, such as germline variation (FIG. 1A), somatic mutations, and allele-specific epigenetic events. Normal cis-regulatory variation is known to be a major determinant of allelic imbalance of expression, affecting a vast majority of the human genome and generating the wealth of phenotypic intraspecies variation. Cis-regulatory variants alter the activity of cis-regulatory elements by affecting protein binding to the DNA, protein RNA, and miRNA. The most robust approach to detect their effect and to map them is by analysing differential allelic expression (DAE) in heterozygous individuals for transcribed polymorphisms. PIK3CA, one of the most frequently mutated genes in breast cancer, has been shown to display mutant allele imbalanced expression in a small percentage of lung cancers, with copy number gain being the most frequent change associated with the increased expression.

Based on the above-mentioned state of the art, the objective of the present invention is to provide means and methods to determine the allelic expression of a mutant and a wildtype allele of the PIK3CA gene in a tumour sample obtained from a heterozygous cancer patient subject, in order to predict their prognosis, and/or sensitivity to certain drugs. This objective is attained by the subject-matter of the independent claims of the present specification, with further advantageous embodiments described in the dependent claims, examples, figures and general description of this specification.

### SUMMARY

The examples presented herein demonstrate the clinical impact of differential expression allelic expression in tumours harbouring somatic PIK3CA mutations. DNA and RNA sequencing data from two independent sets of breast tumours totaling 255 breast cancer cases shows frequent preferential expression of the mutant allele compared to healthy controls. The differential expression of mutant alleles can be associated with different clinicopathological variables of breast tumours or define a subset of patients with different rates of survival.

A first aspect of the invention provides a method of stratifying patients diagnosed with cancer bearing a PIK3CA mutation according to their prognosis, comprising determination of the expression level of both a wildtype and mutant allele of the PIK3CA gene present in a tumour sample, and assigning different clinical outcome based on the relationship of these two values.

Another aspect of the invention uses the method specified above to identify breast cancer patients having a high likelihood of responding favourably to treatment with a phosphoinositide-3-kinase (PIK3) inhibitor drug, due to imbalanced expression of the mutant PIK3CA allele.

The invention further teaches the use of drugs targeting the mutant PI3K protein in a subset of patients which are determined to express a mutant PIK3CA allele above a certain threshold, in addition to a kit, or system with which PIK3CA allelic expression may be assessed.

### BRIEF DESCRIPTION OF THE DRAWINGS

FIGS. 1A-1D show cis-regulatory variation of PIK3CA gene expression in normal breast tissue. FIG. 1A Schematic of cis-regulatory SNP (rSNP) generating DAE on a target gene. Different alleles of an rSNP may have differential binding affinities to transcription factors (red), generating differences in the expression of both alleles, the effect of which can be measured in heterozygous individuals for the daeSNP. FIG. 1B DAE ratios for six daeSNPs in the PIK3CA gene region, each dot is a heterozygous individual for the corresponding variant indicated in the x-axis, dotted lines delimit the 1.5 fold difference for preferential allele expression (|DAE|=0.58); FIG. 1C rs2699887 is significantly associated with DAE for rs12488074. Indicated p-values correspond to Student's t-test test, with 1000 permutations; FIG. 1D Genomic view of the location of rs2699887, showing methylation of surrounding CpG sites (MeDIP-seq), typical active promoter chromatin modification marks (H3K4me3, H3K9ac, H3K36me3) and open chromatin status (DNAseI hypersensitivity and Chromatin Accessibility p-val), as obtained from the Roadmap of Epigenomics project for breast HMECs.

FIGS. 2A-2D show Mutant allelic imbalance in gene expression of somatic missense PIK3CA mutations is frequent in breast tumours, particularly for preferential expression of the mutant allele. FIG. 2A—Schematic representation of the hypothesis, in which cis-acting regulatory variants (rVar), either from germline or somatically acquired, generate different relative allelic expression of mutant and wild-type alleles, resulting in tumours of different prognosis. FIG. 2B—Distribution of α, β and γ ratios in breast tumours. Tumours with MADE, |γ|≥0.58, are displayed in red. FIG. 2C—Correlation analysis of α vs β and α vs γ, showing that both genomic copy-number dosage and allelic expression regulation contribute to allelic imbalances in the expression of mutations in tumours. FIG. 2D—Comparison of matched β and γ values, showing predominance of tumours with preferential allelic expression of the mutated allele, albeit higher number of wild-type allele copies.

FIGS. 3A-3D show survival and clinicopathological effect of mutant allele preferential expression. FIG. 3A Kaplan-Meier curves of disease-specific survival showing the worse prognosis of patients with differential expression of the PIK3CA mutation (MADE group, shown in yellow) compared to those expressing equimolar levels of mutation and wild-type alleles (mut=wt group, shown in blue), in METABRIC. Shown below the graph are the number of patients at risk per group throughout time. FIG. 3B Disease-Specific survival Kaplan-Meier curves with the MADE group subdivided into those with preferential expression of the mutant allele (MADE_mut, shown in red) and those with preferential expression of the wild-type allele (MADE_wt, shown in green), confirming worse survival compared to the mut=wt group (shown in blue), in METABRIC. Shown below the graph are the number of patients at risk per group throughout time. FIG. 3C Preferential expression of the mutated allele is associated with ER negative, PR negative and Her2 positive breast tumours. In all graphs, samples were coloured according to the MADE classification. P-values indicated correspond to Wilcoxon rank sum test with continuity correction, corrected for multiple testing using the Bonferroni correction. FIG. 4D—Kaplan-Meier curves showing the worse disease-specific survival of the MADE groups compared with the mut=wt group, in the ER positive, PR positive and Her2 negative subtypes (ER=oestrogen; PR=progesterone; Her2=human epidermal growth factor 2 receptor) in METABRIC.

FIG. 4 shows that rs2699887 is an eQTL for the expression of PIK3CA in tumours from METABRIC. P value indicated corresponds to Student's t-test.

FIG. 5 shows association analysis between a ratios and ER, PR and HER2 statuses. Boxplots for α ratios for the METABRIC (top) and TCGA (bottom) tumours. Each dot represents a sample and number of samples in each group are indicated in brackets. In all graphs, samples were coloured according to the MADE classification. P-values indicated correspond to Wilcoxon rank sum test with continuity correction, corrected for multiple testing using the Bonferroni correction.

### DETAILED DESCRIPTION

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of”.

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

The term gene refers to a polynucleotide containing at least one open reading frame (ORF) that is capable of encoding a particular polypeptide or protein after being transcribed and translated. A polynucleotide sequence can be used to identify larger fragments or full-length coding sequences of the gene with which they are associated. Methods of isolating larger fragment sequences are known to those of skill in the art.

The term genotype in the context of the present specification relates to the copy number of either a wildtype or a mutant allele present for a specific gene or genetic locus. In other words, whether a sample, or patient, is heterozygous or homozygous for a particular nucleic acid sequence, for example a mutated PIK3CA gene. A homozygous cancer patient sample has two identical alleles, for example two wildtype, unmutated PIK3CA alleles, whereas a heterozygous sample has alleles of more than one type, a wildtype and a mutant allele.

The terms gene expression or expression, or alternatively the term gene product, may refer to either of, or both of, the processes—and products thereof—of generation of nucleic acids (RNA) or the generation of a peptide or polypeptide, also referred to transcription and translation, respectively, or any of the intermediate processes that regulate the processing of genetic information to yield polypeptide products. The term gene expression may also be applied to the transcription and processing of a RNA gene product, for example a regulatory RNA or a structural (e.g. ribosomal) RNA. If an expressed polynucleotide is derived from genomic DNA, expression may include splicing of the mRNA in a eukaryotic cell. Expression may be assayed both on the level of transcription and translation, in other words mRNA and/or protein product.

The term phosphoinositide-3-kinase, catalytic, alpha polypeptide (PIK3CA) in the context of the present specification relates to the PIK3CA gene (ENSG00000121879) encoding the phosphatidylinositol kinase 3 protein p110 alpha subunit (UniProt No. P42336). As mutations within regulatory elements outside of the gene, regulatory intronic regions, or within the 5′ and 3′ untranslated regions are shown to influence allelic expression of the PIK3CA mRNA, the region within a 250 Kb upstream or downstream of the PIK3CA open reading frame are considered to be encompassed within the term PIK3CA, or PIK3CA gene for the purpose of the invention.

The term wildtype or wildtype PIK3CA allele according to the invention refers to the nucleic acid sequence of the PIK3CA gene lacking mutations (ENSG00000121879).

The term PIK3CA mutation, PIK3CA mutant, or variant in the context of the present specification relates to a variant or alteration of the sequence within the transcribed PIK3CA gene locus, The Ensembl database entry for the PIK3CA gene currently lists 10 known splice variants, and 344 known mutant PIK3CA allele nucleic acid sequences. PIK3CA mutations of particular relevance to the invention are those present in a PIK3CA mRNA transcript linked to cancer phenotypes, which are generally missense mutations conferring changed transcription, translation, or activity of the PIK3CA oncogene. Most known PIK3CA mutations are single nucleotide polymorphisms (SNP).

The term Single nucleotide polymorphisms (SNP) in the context of the present specification relates to a single nucleotide that differs from a wildtype sequence, for example, a single nucleotide difference from the wildtype PIK3CA gene specified above. The term encompasses a variant, or mutant detected at a single nucleic acid in the PIK3CA DNA open reading frame, or regulatory elements within 250 kb of the gene, or in an mRNA transcribed from said nucleic acid position. SNP of particular interest according to the invention are specified in the methods of differential gene expression analysis provided in the examples. SNP of particular utility as biomarkers according to the invention, are regulatory SNP such as rs2699887 that are linked to allelic imbalance of PIK3CA, the 6 differentially expressed SNP identified in Table 1, and those in close linkage disequilibrium with the SNP in Table 1 such as those depicted in Tables 3 and 4, which all contribute to differential allelic expression at the PIK3CA locus harnessed for its predictive power for patient outcomes according to the invention.

The term allele, in the context of the present specification relates the two copies of a genetic locus present at within the genome. As diploid organisms, humans have two alleles at most locations, one inherited from each parent, but somatic mutations in an allele may also arise later in life. Heterozygous PIK3CA alleles where one copy of the genetic locus comprises a variation, or mutation, that differs from the wildtype sequence are of particular importance for the methods according to the invention. A mutant, or variant allele, comprises, for example a mutation such as an SNP at a particular genetic locus in the genome. For example, one example of a mutant PIK3CA allele relevant to the invention is a copy of the PIK3CA gene bearing an SNP associated with poor prognosis in breast cancer.

The term mutant PIK3CA allele encompasses to variants or mutations within cis-regulatory elements of the PIK3CA mRNA transcript within introns, or untranslated regions, that are linked to pathogenic expression, and therefore can be utilised as supplemental or stand-alone biomarkers for severe forms of breast cancer.

The term allele-specific expression, or allelic expression is a measure of gene expression that is attributed to one PIK3CA copy, or allele at a specific genetic locus. Allelic expression depends on genotype i.e. whether a subject is homozygous, or heterozygous at a specific nucleic acid region or single base pair position, as well as copy number i.e. relative number of copies of each allele, and other forms of regulation that may affect the transcription rate of one, or both copies of the gene. Allelic expression can be defined as a ratio, or relative expression comparing matched alleles, most often a mutant and wildtype allele at the same genetic locus in a heterozygous individual.

Allelic imbalance or allele differential expression, in the context of the present specification refers to two alleles of a genetic locus in a heterozygous individual which are expressed at a different rate. A genetic locus can be said to exhibit allelic imbalance if one allele is expressed more than would be expected based on copy number, which can be calculated by normalising the expression level of each allele to the amount expected based on the genotype, or copy number in the genome. For example, when considering the total amount of PIK3CA mRNA transcripts in a cell, or tumour sample obtained from heterozygous subject, in the absence of allelic imbalance, half of the total PIK3CA mRNA present will be derived from the mutant PIK3CA allele, and half from the wildtype PIK3CA allele. In other words, the allelic expression level determined for each copy of the PIK3CA gene should be roughly equivalent, as there is one copy of each in the cell. However, if significantly more of than half of total PIK3CA mRNA transcripts of a genetic locus is derived from one allele, this is defined as PIK3CA allelic imbalance.

Mutant allelic imbalance or mutant allele differential expression, MADE, in the context of the present specification refers to a mutant allele, particularly a mutant PIK3CA allele that is expressed at a higher level than its wildtype counterpart. In other words, a form of allelic imbalance as specified above, wherein mutant allele transcripts, or mutant allele proteins outnumber their wildtype counterparts.

As used herein, the term pharmaceutical composition refers to a compound of the invention, or a pharmaceutically acceptable salt thereof, together with at least one pharmaceutically acceptable carrier. In certain embodiments, the pharmaceutical composition according to the invention is provided in a form suitable for topical, parenteral or injectable administration.

As used herein, the term pharmaceutically acceptable carrier includes any solvents, dispersion media, coatings, surfactants, antioxidants, preservatives (for example, antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington: the Science and Practice of Pharmacy, ISBN 0857110624).

As used herein, the term treating or treatment of any disease or disorder (e.g. cancer) refers in one embodiment, to ameliorating the disease or disorder (e.g. slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. Methods for assessing treatment and/or prevention of disease are generally known in the art, unless specifically described hereinbelow.

In spite of the high frequency of PIK3CA mutation in breast cancers, the response to PI3K inhibitor therapy has not been as successful as expected, and the prognostic significance of somatic PIK3CA mutation expression in breast tumour is unclear. Although PIK3CA mutations are known to associate favourably with clinical outcomes and treatment responses, specifically in HR+ breast cancer, the inventors find that not all PIK3CA somatic mutations, are equally expressed in tumours, with some hardly expressing the mutations identified at the DNA level. The findings herein reveal the importance of assessing the expression levels of mutant PIK3CA alleles as part of clinical management.

A first aspect of the invention is a method of predicting the prognosis of a cancer patient, by determining the allelic expression of a wildtype and a mutant PIK3CA allele of a tumour disease characterised by a PIK3CA mutation.

The term tumour disease according to this aspect of the invention encompasses neoplastic cells characterised by at least one PIK3CA mutation. Tissue-derived cancers are of particular relevance for analysis by this method, as PIK3CA mutations are frequently observed across a broad range different tumour types for example epithelial cell-derived cancers such as melanoma, lung, and endometrial cancer (Morgese F. 2014, Oncotarget, 8:75914; Li Q. 2013, Clin. Cancer Res, 19:6252; Wang L., 2012, J. Cancer Res. Clin. Oncol. 138:377).

Certain embodiments of this aspect of the invention relate to a tumour sample, such as a biopsy, or a tissue sample comprising malignant cells, for example a lymph node draining a tumour site.

Particular embodiments relate to a sample obtained from a patient suspected to suffer from, or having been diagnosed with breast cancer, such as a biopsy, or a site of breast cancer metastasis such as an excised lymph node metastasis. The PIK3CA expression status according to these embodiments provides information on the allelic expression of both heritable and somatic mutations existing in cancer cells.

The method according to this aspect of the invention comprises performing the following steps on a tumour sample comprising genetic material obtained from said patient.

In a first measurement step, the following values are determined in said sample:
i. the expression level (ex) of a mutant (mut) PIK3CA allele (ex(mut)), and
ii. the expression level of a wildtype (wt) PIK3CA allele (ex(wt)),
wherein the mutant PIK3CA allele may be any mutation in the PIK3CA gene, particularly an SNP located within the PIK3CA gene such as those listed in Table 1, 3, and 4 of the examples.

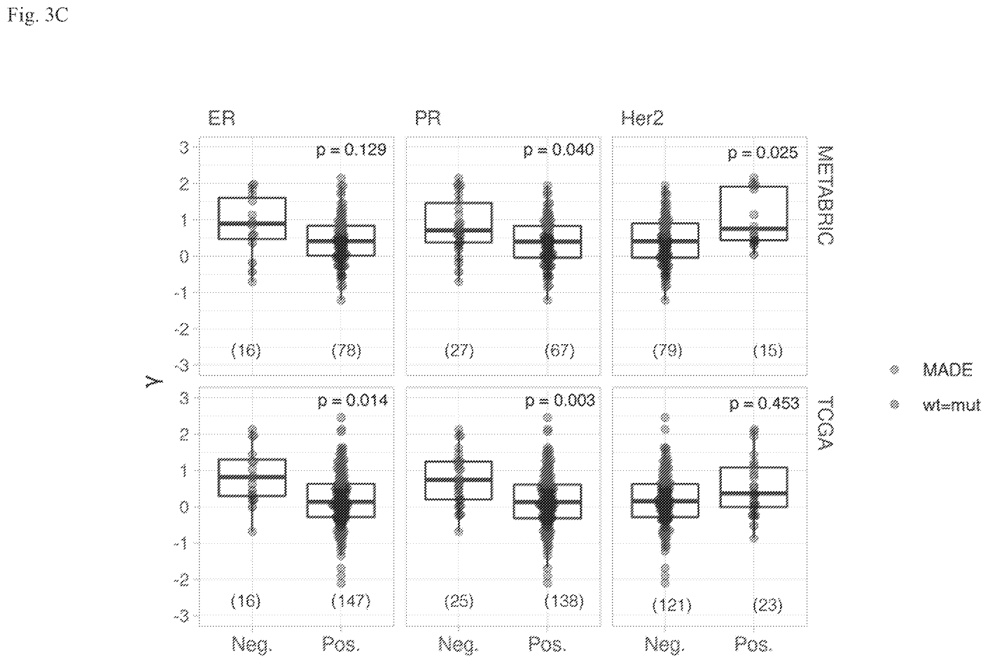

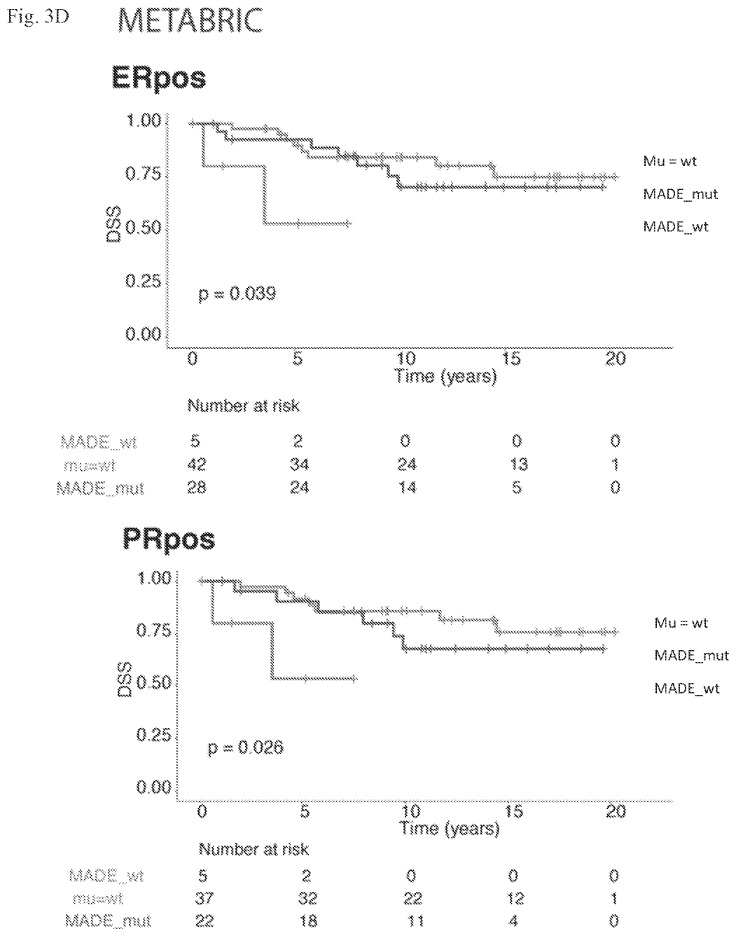

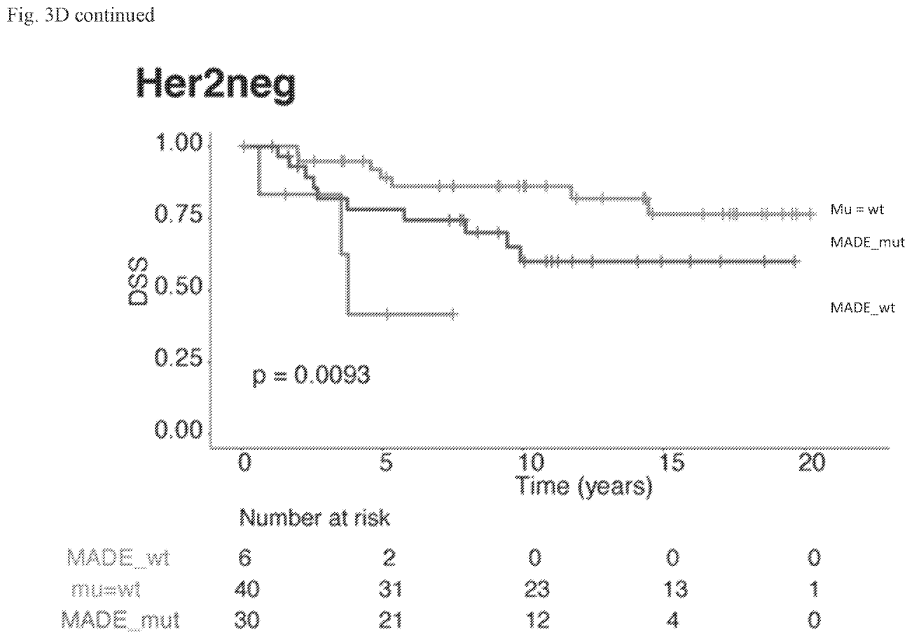

In an assignment step, the expression levels of the wildtype and mutant PIK3CA alleles are compared to provide evidence of imbalanced, or differential allelic expression, when either a mutant or wildtype allele is significantly overexpressed relative to the other. A ratio of wildtype to mutant PIK3CA allele expression is calculated (an allelic expression value), expressed as a fold change value, and this value is used to assign patients a clinical outcome. An allelic expression, or fold change of 1 indicates equivalent level of wildtype and mutant allele expression. A threshold for an allele being significantly overexpressed compared to another, indicating allelic imbalance, must be able to accurately discriminate between patient groups with different PIK3CA allelic expression correlated with distinct survival outcomes based on statistical methods such as those demonstrated in FIG. 3 of the examples. A range, or threshold delimiting significant difference in the expression level of the two alleles being compared must be determined bearing in mind the error rate of the measurement method used, and the range of ratios present in a cohort of patient samples from the same disease with known outcomes.

According to this aspect of the invention the patient is assigned a likelihood of a poor prognosis if the expression level of either the wildtype or mutant PIK3CA allele is significantly higher than the expression level than the other, particularly wherein the wildtype PIK3CA allele is expressed significantly more than the mutant PIK3CA allele, in other words if allelic imbalance is observed at the PIK3CA locus.

Certain embodiments relate to assigning a prognosis to patient with equivalent expression of PIK3CA mutant and wildtype alleles, where poor prognosis is defined as about 45-55% or less probability of survival at about 11 years, and good prognosis is defined as about a 75-85% probability of survival at about 18 years.

Another aspect of the invention relates a method comprising measuring the expression level of a wildtype and mutant PIK3CA allele as specified in the first aspect of the invention, and use of a threshold of a 1.5 fold difference in expression of the mutant or wildtype PIK3CA allele, to assign a patient as having significant allelic overexpression, or imbalance for the purpose of assigning prognosis according to the invention.

A patient whose tumour sample is determined to be characterised by an imbalance in allelic expression, wherein either the mutant or wildtype PIK3CA is expressed at least 1.5 fold more than the other, is assigned a poor prognosis according to this aspect of the invention, particularly wherein the wildtype allele is expressed at least 1.5 fold more than the mutant. Patients with a tumour characterised by a lack of PIK3CA allelic imbalance according to this aspect of the invention expressing each PIK3CA allele equally, wherein the wildtype and mutant allelic expression levels are less than 1.5 fold different from one another, are assigned a better clinical outcome for breast cancer compared to the poor prognosis allelic imbalance groups specified above.

Some embodiments of this aspect of the invention particularly relevant to breast cancer relate to assigning a patient expressing levels of a wildtype and mutant PIK3CA allele less than 1.5 fold different from each other in a tumour sample a good prognosis, wherein good prognosis is defined as about a 75-85% probability of survival at about 18 years.

Some embodiments of this aspect of the invention particularly relevant to breast cancer, relate to assigning a patient whose tumour sample has been determined to have 1.5 fold or more expression of either mutant or wildtype PIK3CA allele, particularly the mutant allele, a poor prognosis which is defined as about a 50-60% probability of survival at about 11 years.

In some embodiments of this aspect of the invention, a patient in which the tumour sample is determined to exhibit overexpression of the wildtype allele, particularly a threshold of 1.5 or more times the level of the PIK3CA mutant allele, is assigned a very poor prognosis, as this patient subset was demonstrated to have the worst overall outcomes in breast cancer.

Some embodiments of this aspect of the invention particularly relevant to breast cancer relate to assigning a patient with a tumour sample assessed to overexpress a wildtype PIK3CA allele compared to a mutant allele a poor prognosis, where poor prognosis is defined as about a 45-55% probability of survival at about 5.5 years.

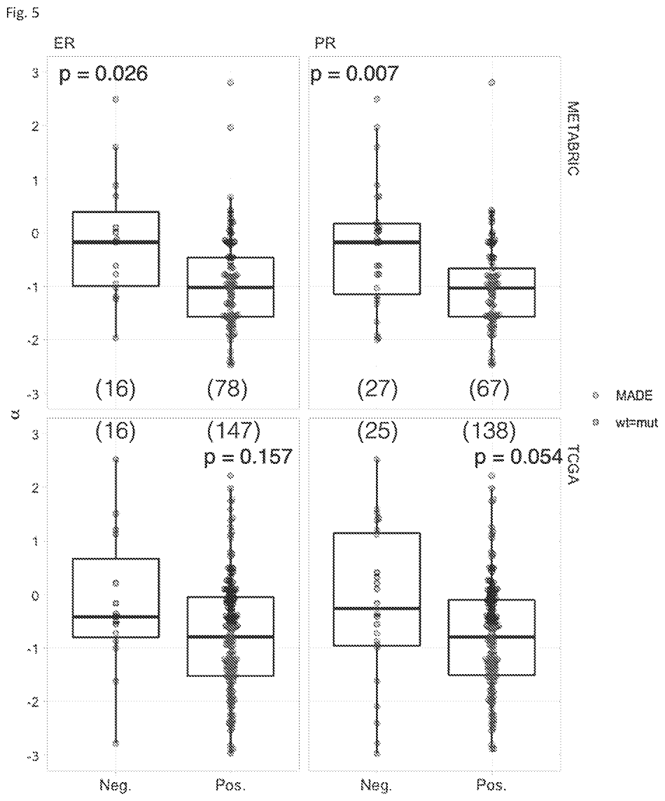

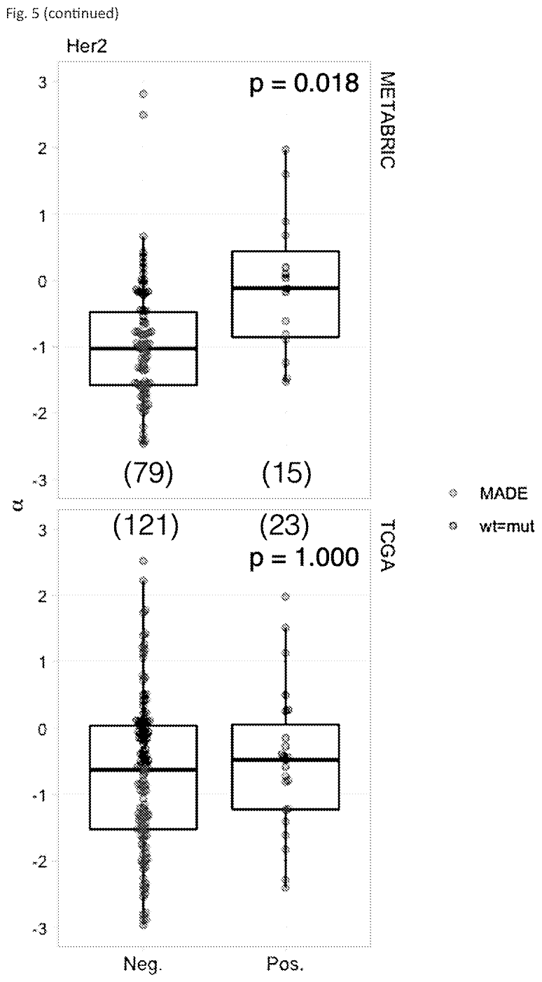

FIG. 5 of the examples demonstrates that overexpression of the mutant PIK3CA allele is associated with poor prognosis compared to patients where each allele is expressed at equivalent levels, and overexpression of the wildtype PIK3CA allele is associated with even worse patient survival, based on a threshold of 1.5 fold as a definition of allelic imbalance.

The skilled practitioner will appreciate that an appropriate threshold for over expression may be identified by analysing a patient cohort according to the method used to assess allelic imbalance in breast cancer tumour samples in the examples, and the threshold may differ slightly depending on the mutations that are measured, and the methodology chosen to measure PIK3CA allelic expression levels according to the invention.

In some embodiments, the threshold for significant allele overexpression, or allelic imbalance according to the invention is a range encompassing statistically equivalent expression of the two alleles, such as the range calculated from a cohort of comparable breast cancer samples in the examples. In alternative embodiments, the sample is compared to control reference samples previously determined to represent the limits of this range.

In one embodiment of the above aspects of the invention, the mutant PIK3CA allele is a measured at the level of the full mRNA transcript comprising a certain PIK3CA mutation.

In other embodiments of the method according to the aspects of the invention above, the PIK3CA mutation expression level is assessed at the level of a single base alteration, in other words a SNP.

Each SNP in Table 1 was assessed for a relationship with breast cancer patient outcomes, and each was shown to exhibit allelic imbalance in certain patients. Combining allelic expression information from more than one SNP is possible in cancer samples which are heterozygous for more than one PIK3CA mutation, or when the expression level of SNP located within both exonic and intronic regions is being assessed.

In some embodiments of the method according to the aspects of the invention specified above, the assay to determine a PIK3CA allele expression level is performed on a tumour sample that has not previously been determined to comprise a mutant PIK3CA allele, as part of the process of screening for the presence of PIK3CA mutant alleles. This may be done by performing an assay which assesses the expression level of more than one mutant PIK3CA allele simultaneously, for example using a microarray to assess the expression level of transcripts comprising different SNP as demonstrated in the examples.

Another embodiment of the method according to aspects of the invention above relates to the use of any quantitative allele-specific measurement of mRNA expression levels, for example mRNA sequencing of the PIK3CA gene and quantification of transcripts which bear either the wildtype or a mutant PIK3CA sequence, or by using primers suitable for polymerase chain reaction amplification of a specific locus characterised by a mutant or wildtype allele.

Another aspect of the invention provides a method for determining the likelihood of responsiveness of a cancer patient's, particularly a breast cancer patient's tumour disease to treatment with a phosphoinositide-3-kinases (PIK3) inhibitor drug. This method identifies patient which overexpress a mutant PIK3CA allele, and are therefore more likely to express the mutant protein targeted by these drugs.

The term, PIK3 inhibitor drug or PIK3 inhibitor therapy according to this specification refers to pharmaceutical compounds which inhibit Class I phosphoinositide-3-kinases comprising the alpha subunit encoded by the PIK3CA gene. PIK3 inhibitors of particular utility for the purpose of the invention, include, but are not limited to, compounds such as alpelisib (Piqray), which targets alpha subunit mutations, or copanlisib (Aliqopa), which targets alpha and beta subunit mutations.

These drugs target mutant variants of the PI3K protein, and therefore will only work if the mutant protein is transcribed, which the inventors find is not the case in all breast cancer patients. The inventors consider it likely that tumours which overexpress a mutant PIK3CA allele have a greater likelihood of response to said chemotherapy treatments in comparison to heterozygous patients who express wildtype and mutant allele equally, and an even greater likelihood of response compared to patients who express relatively little mutant allele.

In a first measurement step, allelic expression of a mutant and wildtype PIK3CA allele is assessed according to the first aspect of the invention. In other words, the expression level is obtained for the mutant, and wildtype PIK3CA gene alleles in a patient sample using a quantitative allele-specific methodology to compare the relative mutant and wildtype transcript abundance.

In a second assignment step, the subject is assigned a likelihood of responsiveness of a cancer patient's, particularly a breast cancer patient's tumour disease to treatment with a mutant PI3K inhibitor drug, if the mutant PIK3CA allele is significantly overexpressed when compared to the wildtype allele at that locus, particularly wherein the mutant PIK3CA allelic expression value is more than 1.5 fold that of the wildtype allele.

Establishing a threshold of statistical significance between PIK3CA expression status values is demonstrated in the examples studying a patient outcomes and allelic imbalance in a cohort of similar patients. The exact value of the threshold is understood to be subject to change as more samples are analysed, or if a method other than mRNA microarray is used to measure allelic imbalance according to the invention.

This method identifies those patients in which mutant PIK3CA allele is expressed at the level of mRNA, and uses the value to stratify patients into groups which are likely to have different outcomes from PIK3 inhibiting medications as specified above. Patients assigned a higher likelihood of responsiveness here include both those patients that display a PIK3CA expression status value within the range that denotes equal expression of the wildtype and mutant allele, and also those patients in which the mutant allele is over expressed. These two groups, particularly the latter, are predicted to respond to drugs targeting mutant better than those with low mutant PIK3CA mRNA expression, resulting in low protein expression of the mutant allele. This information could help a clinician to identify patients who are likely to benefit from PI3K-targeted chemotherapy treatment (increased overall or disease-specific survival).

The method by which the expression levels of PIK3CA alleles are measured at the mRNA level is not limited according to the invention, and includes the use of allele-specific nucleic acid probes, particularly with a quantitative PCR methodology such as real time PCR, or qPCR, sequencing reactions, or a nucleic acid array. In other embodiments, the PIK3CA expression status is determined by mRNA sequencing.

One methodology that is particularly useful for measuring the biomarker PIK3CA nucleic acid allelic expression level, is a nucleic acid amplification method conducted using polymerase chain reaction of the RNA extracted from the patient tumour sample. The term cycle threshold in the context of the present specification relates to a quantitative nucleic acid measurement, for example a measurement made with a quantitative polymerase chain reactions (qPCR). This method involves repeated cycles of nucleic acid amplification using nucleic acid probes which hybridise a target wildtype and mutant allele, to generate a product emitting a fluorescent signal, which can be measured to determine the amount of starting genetic material. The cycle threshold may be an average value, or the average value of a number of replicate samples. Other quantitative measurements may substitute the cycle threshold, such as a crossing point, or an adjusted inflexion point.

The skilled artisan will appreciate in embodiments wherein a PIK3CA allele variant expression is measured by qPCR, values may be expressed examples of differential cycle thresholds compared to a house keeping gene, i.e. the number of qPCR cycles needed to generate a fluorescence signal from the specific nucleic acid probes used, above a user-defined threshold.

These values therefore reflect the PCR conditions and cycle threshold, and the exact values of a threshold for pathogenic expression may expected to vary from those derived in the examples from mRNA sequencing samples. Similar thresholds relevant for breast cancer outcomes for qPCR measurements of allelic expressing can be generated by assessing a cohort of similar samples using the same methodology, and performing a correlation analysis with patient outcomes, as demonstrated in the examples. Specific nucleic acid probes can identify expression of a PIK3CA variant with a primer designs which comprise the complementary sequence to either a WT, or a SNP region, or allele, using for example, standard TAQman ABI or Sybrgreen enzyme qPCR assay conditions.

In another embodiment of the method to predict the prognosis, or the drug response of a cancer patient according to the aspect specified above, the allelic expression level of the mutant and wildtype PIK3CA copies is assessed by mRNA sequencing technology, or an mRNA microarray assay, such as the Illumina array utilised in the examples. These methodologies allow non-targeted assessment of any mutant alleles present in a patient sample, as patients may be heterozygous for more than one SNP.

In another embodiment of the method to establish the prognosis, or the drug sensitivity of a cancer patient according to aspects of the invention specified above, the expression level of mutant PIK3CA allele is determined for at least one SNP locus selected from rs7636454, rs3960984, rs3729679, rs2699887, rs12488074, rs4855093, and/or rs9838411. Some SNP are relatively rare, and will only be detected infrequently in patients, thus in embodiments where the mutation status of the patient sample is not yet known, preferably the expression level of mutant PIK3CA allele is determined several common SNP loci, or at all listed loci.

In particular embodiments of the aspects of the invention described above, the expression level of mutant and wildtype PIK3CA allele is determined for a common SNP mutation found in more than half of patients with a mutant PIK3CA gene, such as the SNP locus rs3960984. Measuring the expression level of the SNP rs3960984 and its wildtype counterpart allele is particularly useful according to the invention due to both its high prevalence in PIK3CA mutant breast cancer samples, in addition to the high occurrence of imbalanced allelic expression of mutant and wildtype rs3960984 alleles (Table 1). Other known SNP that are similarly common among cancer patients are particularly desirable for use according to the invention.

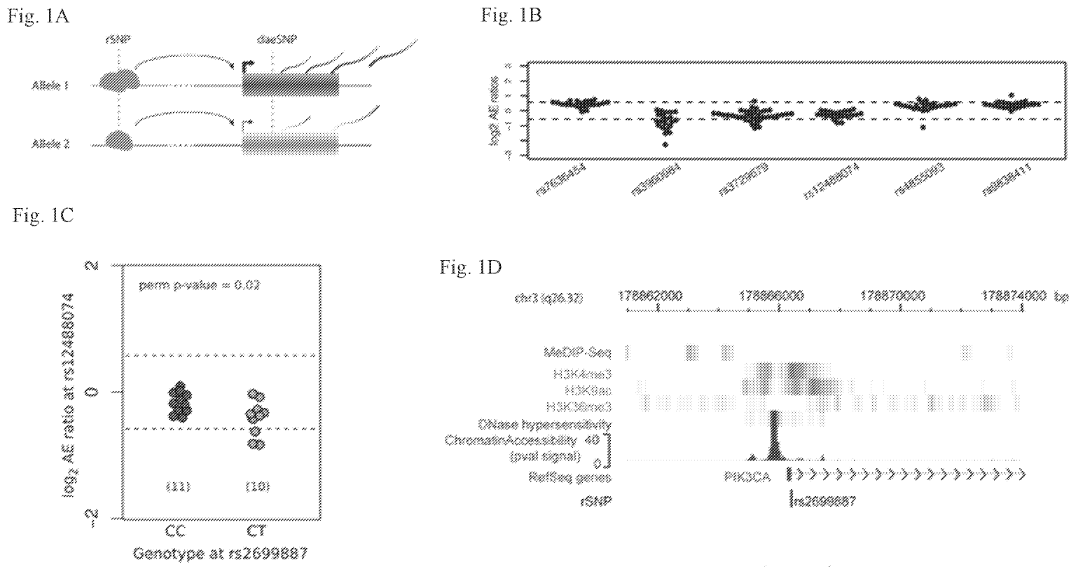

In another embodiment of the aspects of the invention specified above, the expression level of mutant and wildtype allele is determined at a SNP locus located within untranslated region of the PIK3CA transcript, particularly wherein the expression level of mutant PIK3CA allele is determined for the SNP rs2699887. Allelic imbalance of several mutant PIK3CA allele rs12488074 is demonstrated to be influenced by the presence of the cis-regulatory SNP locus rs2699887 in FIG. 1 of the examples.

Particular embodiments of the methods specified above relate to predicting prognosis or tumour-sensitivity to PIK3 inhibitors in patients diagnosed with a form of breast cancer. In some embodiments of the invention according to any of the previous aspects of the invention, the patient has been diagnosed with a breast cancer tumour that has been classified as oestrogen receptor positive, progesterone receptor positive, or human epidermal growth factor receptor 2 negative.

In the data shown in the examples, preferential expression of a mutant PIK3CA allele is associated with known poor prognosis variables, such as PR negative and Her2 positive statuses. In the METABRIC dataset, the inventors found that patients with tumours with mutant allelic expression imbalance had worse overall survival than those without it, particularly in the ER positive, PR positive and Her2 negative subsets. The method according to the invention can deliver additional information compared to current standard-of-care prognostic histology markers, by identifying subset of patients with differing survival outcomes (FIG. 3).

Another aspect of the invention relates to a system enabling the method as described above. The system according to the invention comprises a device for obtaining expression data from tumour cells, a sequencing apparatus for generation of PIK3CA allele expression data, and a device for electronic processing of such PIK3CA allele expression data according to the invention as disclosed herein.

### Pharmaceutical Compositions and Administration

Another aspect of the invention relates to a pharmaceutical composition comprising a PIK3 inhibitor drug selected from alpelisib, or copanlisib, or a pharmaceutically acceptable salt thereof, and a pharmaceutically acceptable carrier. In further embodiments, the composition comprises at least two pharmaceutically acceptable carriers, such as those described herein.

In certain embodiments, alpelisib or copanlisib, is administered in combination with further cancer medications in a combination treatment, for example, with a selective oestrogen receptor degrader such as fulvestrant.

In certain embodiments of the invention, the compound of the present invention is typically formulated into pharmaceutical dosage forms to provide an easily controllable dosage of the drug and to give the patient an elegant and easily handled product.

The pharmaceutical composition can be formulated for enteral administration, particularly oral administration or rectal administration. In addition, the pharmaceutical compositions of the present invention can be made up in a solid form (including without limitation capsules, tablets, pills, granules, powders or suppositories), or in a liquid form (including without limitation solutions, suspensions or emulsions).

The pharmaceutical composition can be formulated for parenteral administration, for example by i.v. infusion, intradermal, subcutaneous or intramuscular administration.

The dosage regimen for the PIK3 inhibitor compounds of the present invention will vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. In certain embodiments, the compounds of the invention may be administered in a single daily dose, or the total daily dosage may be administered in divided doses of two, three, or four times daily.

In certain embodiments, the pharmaceutical composition comprising a PIK3 inhibitor drug of the present invention can be in unit dosage of about 1-1000 mg of active ingredient(s) for a subject of about 50-70 kg. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

One aspect of the invention relates to a PI3K inhibitor drug, particularly a PI3K inhibitor drug selected from alpelisib, or copanlisib for use in the treatment of a breast patient who has determined to be responsive by a method according to any of the previously described aspects of the invention.

A further aspect of the invention encompasses the use of nucleic acid probes for amplification or/or detection of an expression level of
a. a mutant PIK3CA allele, and
b. a wildtype PIK3CA allele,
   in a kit for analysing biomarkers in order to determine the PIK3CA expression status of patient tumour sample. This kit can be used on patient tumour samples to deliver information to a clinician on the prognosis of a breast cancer patient, or to predict the likelihood that a breast cancer patient will respond favourably to treatment with PI3K inhibitors.

The method may be embodied by way of a computer-implemented method, particularly wherein the evaluation and the assignment step are executed by a computer.

Further, the method may be embodied by way of a computer program, comprising computer program code, that when executed on the computer cause the computer to execute at least the evaluation and/or assignment step. Particularly, the results of the measurement step may be provided to the computer and/or the computer program by way of a user input and/or by providing a computer-readable file comprising information regarding the methylation level obtained during the measurement step. Results from the measurement step may be stored for further processing on a memory of the computer, on a non-transitory storage medium.

### Medical Treatment, Dosage Forms and Salts

Similarly, within the scope of the present invention is a method or treating breast cancer in a patient in need thereof, comprising administering to the patient a PI3K inhibitor drug, particularly a PI3K inhibitor particularly a PI3K inhibitor drug selected from alpelisib (CAS NO 1217486-61-7) or copanlisib (CAS NO 1032568-63-0) according to the above description.

The skilled person is aware that any specifically mentioned drug may be present as a pharmaceutically acceptable salt of said drug. Pharmaceutically acceptable salts comprise the ionized drug and an oppositely charged counterion.

Wherever alternatives for single separable features such as, for example, a PI3K inhibitor or a medical indication are laid out herein as “embodiments”, it is to be understood that such alternatives may be combined freely to form discrete embodiments of the invention disclosed herein.

The invention is further illustrated by the following examples and figures, from which further embodiments and advantages can be drawn. These examples are meant to illustrate the invention but not to limit its scope.
Tab. 1 shows DAE of normal breast samples in six daeSNPs located at the PIK3CA region.
Tab. 2 shows summary of allelic expression imbalance of somatic PIK3CA mutations in breast tumours.
Tab. 3 shows linkage disequilibrium between daeSNPs of PIK3CA. Data was mapped and annotated according to the “grch37/1kgpp3v5” release, European population, using the tool SNiPA (snipa.helmholtz-muenchen.de).
Tab. 4 shows candidates SNPs from mapping analysis of DAE data in PIK3CA.
Tab. 5 shows summary Statistic of PIK3CA's MADE Survival Analysis in TCGA set. Median, LCL and UCL in days; LCL: lower confidence level; UCL: upper confidence level; N: number of people of each group.

### EXAMPLES

### Material and Methods:

### Differential Allelic Expression Analysis

DNA and total RNA from 64 samples of normal breast tissue, were analysed using Illumina Exon510S-Duo arrays (humanexon510s-duo), as described (Liu R., Bioinformatics 2012, 28:1102). After normalization, SNPs with average log 2 RNA intensity values lower than 9.5 and less than 5 heterozygous values were excluded from the analysis. A two samples Student's t-test was applied to compare RNA log ratios between heterozygous (AB) and homozygous groups (AA and BB). Only SNPs with p-values lower than 0.05 for all comparisons were further analysed. The following equation was used for normalisation of DAE: log₂ ((RNA allele A/RNA allele B)/(DNA allele A/DNA allele B)). This analysis was carried out using R and Bioconductor packages as described (Huber W., Nature Pub. Group 2015, 12:115). DAE was inferred when |AE ratio|≥0.58 (1.5 fold or greater difference). This threshold was established based on the sensitivity and specificity of the applied DAE detection method. Linkage disequilibrium (LD) between daeSNPs was evaluated using the genetic variant-centered annotation browser SNiPA (Li Y., Genet Epidemiol. 2010, 34:816).

### Genotype Imputation Analysis on Normal Breast Tissue Samples

Genotype imputation was run on the Illumina Exon 510 Duo germline genotype data from the 64 samples that passed microarrays quality control filters. Prior to imputation data, a quality control was applied to the genotyping data and SNPs with call rates <85%, minor allele frequency <0.01, and Hardy-Weinberg equilibrium with p-value <1.0e⁻⁰⁵ were excluded from the analysis. Genotype data from the chromosome 3 Illumina SNPs that passed quality control was used to impute genotypes at all additional known SNPs in the chromosome using MACH1.0 (Li, Y. et al., Genet. Epidemiol. 34, 816-834, 2010) and the phased haplotypes for HapMap3 release (HapMap3 NCBI Build 36, CEU panel—Utah residents with Northern and Western European ancestry) ©AS reference panel. For imputation with MaCH1.0, a two-step imputation process was used: model parameters (crossover and error rates) were estimated prior to imputation using all haplotypes from the study subjects and running 100 iterations of the Hidden Markov Model (HMM) with the command options: -greedy and -r 100. Genotype imputation was then carried out using the model parameter estimates from the previous round with command options of -greedy, -mle, and -mldetails specified. Imputation results were assessed by the platform-specific measures of imputation uncertainty for each SNP (rsq Score).

MaCH -rsq score equals the ratio of the empirically observed variance of the allele dosage to the expected binomial variance p(1-P) at Hardy-Weinberg equilibrium, where p is the observed allele frequency derived from HapMap or estimated from own data. Its value tends to zero if the uncertainty of the imputation results increases. Imputation results were assessed by the platform-specific measures of imputation uncertainty for each SNP (rq Score) and the results were filtered for an rq Score higher than 0.3 (Li Y., Genet Epidemiol. 34:816-834, 2010).

### Differential Allelic Expression (DAE) Mapping Analysis on Normal Breast Tissue Samples

Differential allelic expression mapping analysis was performed by stratifying AE ratios at each PIK3CA daeSNP according to the genotype at the genotyped/imputed SNPs located within ±250 Kb of the daeSNP. A two-sample t-test was applied to assess differences between the mean AE ratio between the heterozygous group samples and the combined homozygous groups. P-values were corrected for multiple testing using permutation procedure for N=1000. Permutation-corrected p-values were considered significant below 0.05 and when on average the heterozygous samples displayed larger fold differences between alleles when compared to homozygous samples.

### Proxy SNP Query

Proxy SNPs were identified using HaploReg v4.1 available at pubs.broadinstitute.org/mammals/haploreg/haploreg.php, using genotype data from the Phase 1 of the 1000G project, for the CEU population, with a LD threshold of r²>0.8 (Ward L. D., Nucleic Acids Res. 2011, 40:D930; 1000 Genomes Project Consortium, Nature, 2015, 536:68)

### Functional Annotation of Associated Variants

To predict the most likely functional variants, variants associated with AE levels were mapped to epigenetic marks derived from the Encyclopedia of DNA Elements (ENCODE) and NIH Roadmap Epigenomics projects data, such as chromatin states (chromHMM) annotation, regions of DNase I hypersensitivity, transcription factor binding sites and histone modifications of epigenetic markers (H3K4Me1, H3K4Me3 and H3K27Ac) (genome.ucsc.edu/ENCODE/) for normal human mammary epithelial cells (HMECs), human mammary fibroblasts (HMFs), BR.MYO (breast myoepithelial cells) and BR.H35 (breast vHMEC) and for two breast cancer cell lines MCF-7 and T47D. The inventors prioritized variants that were located in either active promoters or enhancer regions in mammary cell lines, and for which ChIP-Seq data indicated protein binding or PWM scores predicted differential protein binding according to the allele.

Two publicly available tools, RegulomeDB and HaploReg v4.1 and the MotifBreakR Bioconductor package were also used to evaluate those candidate functional variants.

### Breast Tumour Samples

The METABRIC dataset of tumour samples included 2433 samples DNA sequencing data, 480 were subjected to a capture-based RNA sequencing study (Curtis C., Nature 2012, 486:346). Sequencing libraries were generated using total RNA generated from frozen tissues with a TruSeq mRNA Library Preparation Kit using poly-A-enriched RNA (Illumina, San Diego, CA, USA) and enriched with the human kinome DNA capture baits (Agilent Technologies, Santa Clara, CA, USA). Six libraries were pooled for each capture reaction, with 100 ng of each library and sequenced (paired-end 51 bp) on an Illumina HiSeq2000 platform. A subset of samples that have both DNA and RNA sequencing data and harbour PIK3CA mutations was selected for further analysis. The TCGA dataset comprised of 695 samples from TCGA breast cancers, a subset of 289 samples with PIK3CA mutations was selected for further analysis.

### DNA-Seq and RNA-Seq Variant Calling in Tumours

Alignment & Preprocessing: Sequence data (FASTQ) mapped to reference genome (hg19) were aligned using STAR v2.4.1c (github.com/alexdobin/STAR). A two-pass alignment was carried out: splice junctions detected in the first alignment run are used to guide the final alignment. Duplicates were marked with Picard v1.131 (picard.sourceforge.net). Indel realignment and base quality score recalibration were performed using the Genome Analysis Toolkit (GATK). Variant Calling and Annotations: SNV and indel variants were called using GATK Haplotype Caller. Variants were filtered by applying hard filters using GATK VariantFiltration. Variants were annotated with Ensembl Variant Effect Predictor (VEP). Heterozygous genotypes were called from DNA data to avoid RNA editing, and other RNA related variants and because true allelic imbalance can lead to heterozygous sites being called homozygous in RNA-based genotype calling.

### Statistical Analysis of Allelic Expression Imbalances in Tumours

Prior to the analysis, a set of filtering steps were performed to select samples which: 1) had passed quality control; 2) harboured missense mutations; 3) and had a minimum of 30 reads for both RNA and DNA data. Clinical data for METABRIC was updated from the original studies with the latest available records. Clinical data for TCGA were imported from portal.gdc.cancer.gov/ in 26 Nov. 2018. For all samples three parameters were calculated: 1) The DNA mutant allele ratio β=Log₂[(mutant allele read count in DNA)/(wild-type allele read count in DNA)], which served to control for sequencing artefacts from heterozygous genotypes and to account for differences in variant frequencies in DNA; 2) α=Log₂[(mutant allele read count in RNA)/(wild-type allele read count in RNA)], that served as a measure of the net allelic expression imbalance in the tumours; and 3) the normalized mutant allele expression ratio γ=α−β, which is a measure of mutant allelic expression imbalance due to cis-regulation alone. Association between allelic expression imbalance ratios and clinical data was achieved by bivariate analysis Wilcoxon rank sum test with continuity correction or Kruskal-Wallis rank sum test, as indicated in tables and figures. P-values were adjusted per study using the Bonferroni correction and were considered significant when ≤0.05. Correlation analysis “alpha vs beta” and “alpha vs gamma” ratios for both sets of samples were performed using a Pearson's test. All statistical analysis and data visualization were performed using R. Additionally, the inventors considered that there was mutant allele differential expression (MADE) when |γ|≥0.58 (1.5 fold or greater difference). In the survival analysis, the inventors further separated the MADE samples into MADE_wt (γ≤−0.58, or preferential expression of wild-type allele) and MADE_mut (γ≥0.58, or preferential expression of mutant allele).

### Survival Analyses

Kaplan-Meier plots and multivariate Cox proportional hazard models examine the association between allelic expression and survival calculated using the R package Survival. Death due to all causes was used as endpoint, and all subjects still alive were censored at the date of last contact. Kaplan-Meier survival curves were compared using the log-rank test. For the multivariate analysis, Cox proportional hazard model was used to assess the effect of □ on overall survival. Hazard ratios (HRs) and 95% confidence intervals (CI) were estimated by fitting the Cox model while adjusting for age and tumour characteristics, such as size, Scarff-Bloom-Richardson histological grade, clinical stage and oestrogen receptor (ER), progesterone (PR) and human epidermal growth factor 2 (Her2) statuses. All p-values were two-sided, and p-values less than 0.05 were considered statistically significant.

### Code and Data Availability

The filtered data and code for the analysis of mutant allelic expression imbalances and for the survival analysis can be publicly accessed at github.com/maialab/pik3ca.

### Example 1: Normal Cis-Regulatory Variation Affects PIK3CA Expression in Healthy Breast Tissue

To investigate whether the expression of PIK3CA in normal breast tissue was moderated by cis-regulatory variation, the inventors analysed data from allelic expression analysis of normal breast tissue from 64 healthy donors. The inventors calculated the logarithm of the ratio of expression of one allele by another in heterozygous SNP positions (aeSNPs), and differential allelic expression (DAE) when this difference between alleles was greater than 1.5 fold, i.e. the absolute value of log₂ allelic expression ratio ≥0.58. This approach robustly detects cis-acting variant effects, as it cancels out the trans effects that influence both alleles equally. Six SNPs in PIK3CA displayed DAE (daeSNPs), out of 14 tested aeSNPs (FIG. 1B). All daeSNPs, except rs3729679, are in strong linkage disequilibrium (LD) with each other (Tab. 3).

rs3960984 showed the largest proportion of heterozygotes displaying DAE (57%) is, whilst the smallest fraction (14%) is shared by three daeSNPs, rs12488074, rs4855093 and rs9838411. Tab. 2 summarises the DAE findings including the frequency of heterozygotes and the allele preferentially expressed for each daeSNP. The range of allelic expression imbalances detected was between 1.55 and 2.14-fold (DAE ratio between 0.63 and 1.10). In four out of the six daeSNPs (rs7636454, rs3960984, rs12488074, rs9838411) the DAE ratios did not show a bilateral distribution, with all samples with DAE preferentially expressing the same allele. For the other two daeSNPs, only one sample for each differed in the preferentially expressed allele. These patterns of DAE distribution suggest that the daeSNPs and the possible functional regulatory SNPs (rSNPs) are in strong, but not complete, LD with each other (Xiao R., Genet. Epidemiol. 2011, 35:515).

### Example 2: Candidate rSNP for PIK3CA Affects a NF-YA Binding Site at its Promoter

Next, the inventors proceeded to identify the rSNPs acting on PIK3CA by carrying mapping analysis for all daeSNPs, using genotyping information from the SNPs that were located within a 250 kb window from the beginning and end of the PIK3CA gene (NM 006218, GRCh38/hg38). This exercise identified a list of 44 candidate SNPs that, together with their 229 proxy SNPs, significantly explained the DAE observed (Tab. 4).

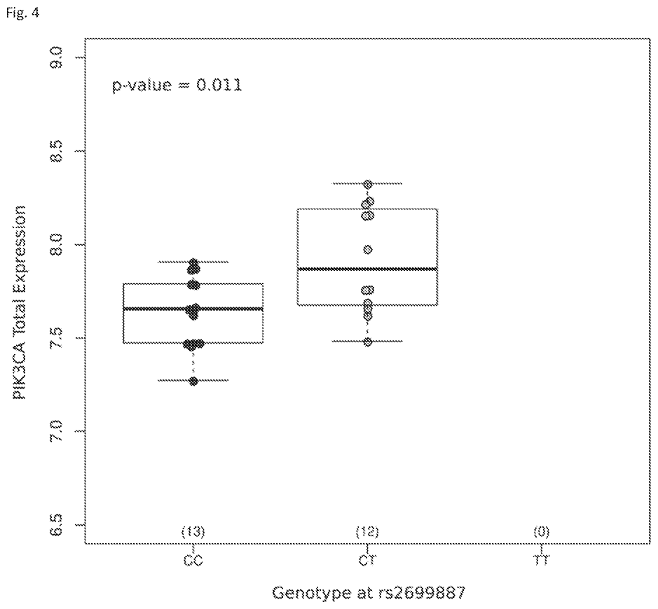

Functional analysis was carried for these 273 candidates, starting with in-silico analysis to identify those with greater potential for being cis-regulatory, followed by in-vitro validation, resulting in one strong candidate rSNP, rs2699887. This variant significantly associated with DAE detected at rs12488074 (permuted p-value=0.02) (FIG. 1C), and associated with differences in the total expression of PIK3CA (p-value=0.012, FIG. 4) in tumours from METABRIC. rs2699887 resides in the first intron of PIK3CA, in a region rich in epigenetic marks and classified as an active promoter in breast mammary epithelial cells (vHMEC) and breast myoepithelial primary cells (FIG. 1D). The motif position weight matrix (PWM) analysis of the sequence surrounding rs2699887 suggested NF-Y proteins could have preferential binding affinity to the minor allele (T allele). Electrophoretic mobility shift assays (EMSA) confirmed the preferential protein-DNA binding potential of the minor allele of rs2699887 (T allele), which upon competition and supershift assays was confirmed to bind preferentially to the transcription factor NF-YA.

### Example 3: Preferential Expression of the PIK3CA Mutated Alleles is Common in Breast Tumours

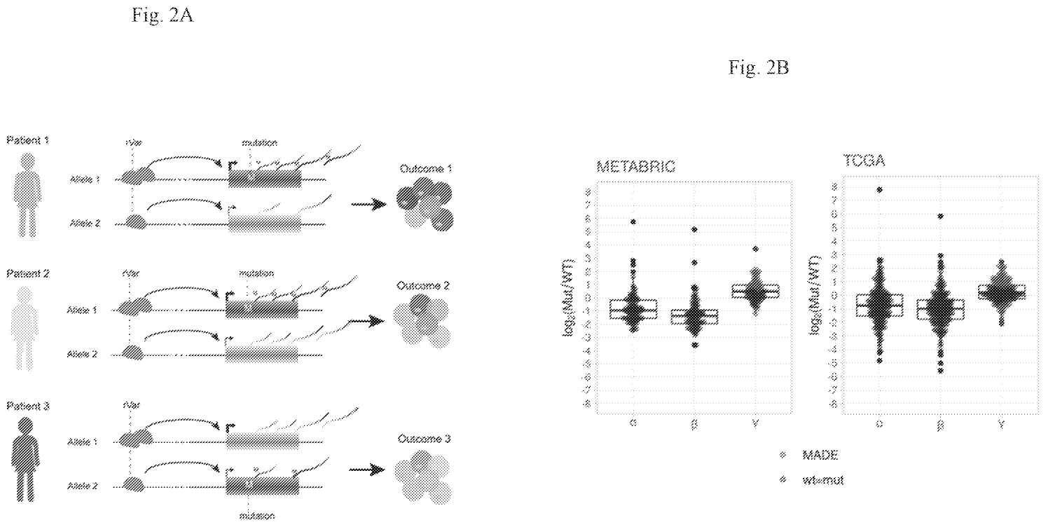

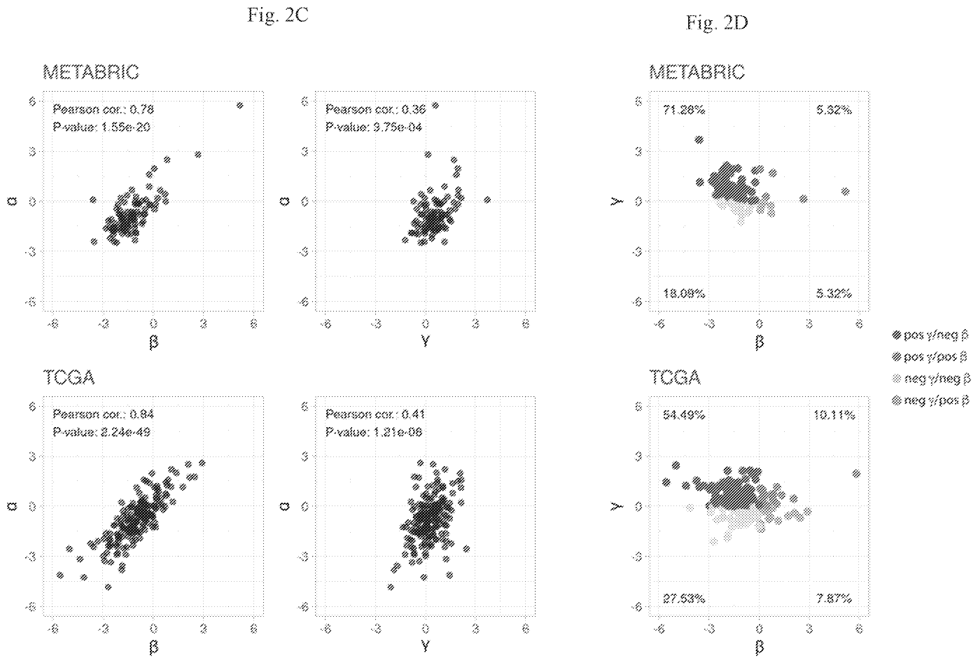

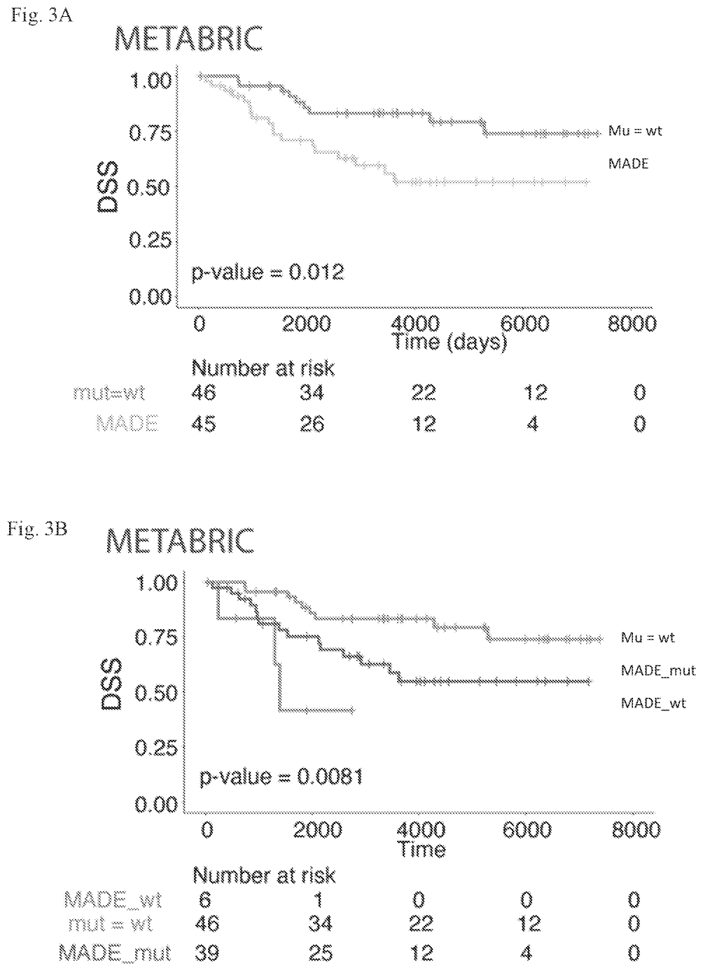

Changes in DNA copy number in tumours have been associated with changes of gene expression in cis, but whether these differences could also be due to cis-regulation of expression has never been analysed. In view of the DAE identified for PIK3CA in normal breast tissue, PIK3CA somatic mutations were assessed for functional effects modified by imbalances in allelic expression. Preferential expression of a gain-of-function mutation may have a stronger clinical impact than lowly expressed alleles, thus generating inter-tumour clinical heterogeneity (FIG. 2A). To test this, mutant vs wild-type allelic expression analysis was performed on breast tumour samples carrying somatic PIK3CA missense mutations from two independent data sets, METABRIC (n=94) and TCGA (n=161). Three allelic ratios from DNA-seq and RNA-seq data were assessed for each mutation:
α=log₂ (number of mutant RNA-seq reads/number of wild-type RNA-seq reads), i.e. the net mutant allele expression imbalance;
β=log₂ (number of mutant DNA-seq reads/number of wild-type DNA-seq reads), i.e. the mutant allele relative copy-number;
γ=α−β, i.e the net mutant allele expression imbalance normalised for the DNA allelic copy-number imbalances, which corresponds to putative mutant allele expression imbalance due to cis-regulation.

α reports the overall allelic expression imbalance derived from different mechanisms including copy-number aberrations, cellularity differences and cis-regulatory variation; whilst γ will report specifically on the contribution from cis-regulatory variants (rVar in FIG. 2A), including normal genetic variation, somatic noncoding mutations and allelic epigenetic changes (FIG. 2B). Mutant allele expression imbalances (a ratio) are frequent in breast tumours (METABRIC (75.5%) and TCGA (68%), Tab. 2). The same is true for γ ratios, at 50.4% for METABRIC and 46% for TCGA, indicating that cis-regulatory effects acting on mutations are also frequent in breast tumours. Samples in both cohorts showed a large values of preferential allelic expression for the mutant allele (maximum 54-fold and 220-fold, in METABRIC and TCGA respectively), but not wild-type alleles (fold differences of 5.5 and 28.2-fold, in METABRIC and TCGA respectively) (Tab. 2, FIG. 2B). Similarly, the trend of more pronounced preferential expression of the mutant allele was found for the γ ratios, 13 and 5.5-fold for METABRIC and TCGA respectively, albeit with smaller fold differences between alleles. Interestingly, samples with mutant allele differential expression (MADE, defined as I-yl a threshold of 0.58) were enriched for preferential expression of the mutated allele in both data sets (binomial test Prob=0.88, CI95%=[0.75-0.95],p=1.0e-07 for METABRIC and Prob=0.70, CI95%=[0.58-0.80], p=7.6e-04 for TCGA).

### Example 4: Cis-Regulatory Variants Contribute Significantly to Imbalances in the Expression of Mutant Alleles

Next, the contribution of each copy number and cis-regulatory variants towards the net mutation allelic expression imbalances detected was assessed in tumours. Overall allelic expression correlated with both copy number and cis-regulatory variation (FIG. 2C), albeit with an effect for the copy number approximately double the size of that found for cis-regulatory variation. The variance of overall allelic expression was considered as the sum of the effects of both mechanisms, plus the covariance accounting for predicted non-mutually exclusion of both the mechanisms acting on any given allele: Var (α)=Var (β)+Var (γ)+2 Cov (β, γ).

The contribution of cis-regulatory variation to the variance of overall allelic expression as: [Var(γ)+Cov(β,γ)]/Var(α).

Cis-regulatory variants accounts for 17.9% and 16.7% of the variability of overall mutant allelic expression seen in METABRIC and TCGA, respectively. Assessment of how the two mechanisms act simultaneously on each tumour showed the majority of samples (71.28% and for the METABRIC and TCGA, respectively) had positive γ and negative β values (FIG. 2D), meaning that although the mutant allele was in lower genomic quantity, it was nevertheless being more strongly expressed than the wild-type allele. Only a small fraction of samples displayed co-occurring preferential allelic expression and higher allele copy number of the mutant allele (5.32% and 9.94% for the METABRIC and TCGA, respectively). A comparison of PIK3CA's matched β and γ values revealed no pattern of association between ratios and cellularity in METABRIC tumours.

### Example 5: MADE Defines an Aggressive Subset of PIK3CA Mutated Tumours

To investigate the impact of differential cis-regulation of PIK3CA's mutations on clinical outcome univariate survival analysis was performed with γ ratios categorised in two groups: the MADE group (when |γ|≥0.58) and the mut=wt group (when |γ|<0.58). The MADE group had poorer overall survival rate (p-value=0.03) and disease specific survival rate (p-value=0.012, FIG. 3A), than the mut=wt group for METABRIC. The median overall survival for the MADE group was approximately 8 years and for the mut=wt group was 18.5 years (FIG. 3A), whilst in the disease-specific analysis 40% of MADE patients died during the length of the follow-up, in comparison with 20% deaths in the mut=wt group (Tab. 5). Significantly shorter overall and disease specific survival was observed for the MADE samples that preferentially expressed the wild-type allele (MADE_wt group, p-value=0.0073 and =0.0081, respectively) in METABRIC (FIG. 3B).

### Example 6: PIK3CA MADE Associates with Clinicopathological Variables

PIK3CA's MADE was compared to current standard-of-care prognostic clinicopathological variables, hormone receptors (ER, PR) and Her2 amplification (FIG. 3C, Tab. 3), which are directly and indirectly connected to gene expression regulation, respectively. For METABRIC, the mean a was significantly higher in ER negative tumours (p-value=0.026), PR negative tumours (p-value=0.007) and Her2 positive tumours (p-value=0.018) (FIG. 5). When evaluating the contribution of cis-regulatory variation to this association, the inventors also found that higher average γ values associated with lower PR expression (p-value=0.040) and Her2 positive tumours (p-value=0.025), but did not find a significant association with ER expression (p-value=0.129) (FIG. 3C). This showed that the predominant abundance of the mutant allele was associated with worse prognosis variables (ER negativity, PR negativity, Her2positivity), particularly in tumours with preferential mutant allele expression. The analysis of the smaller TCGA set corroborated the significant association between the PR status and γ ratios (p-value=0.003) and ER status and γ values (p-value=0.014) (FIG. 3C, FIG. 5). In view of these results, MADE was considered in the survival analysis within the expression subgroups of ER, PR and Her2. MADE vs mut=wt groups did not reveal significant differences in overall and disease specific survival, although there was a trend for worse survival for the MADE category in the Her2 negative subgroup (p-value=0.056 and 0.027 in overall and disease-specific analyses respectively). However, further stratifying the MADE groups revealed a poorer prognosis for MADE_wt samples was identified in ER positive (p-value=0.004), PR positive (p-value=0.0013) and Her2 negative tumours (p-value=0.0066) (FIG. 3D). Thus, MADE defines an aggressive subgroup within the usually good prognosis ER-positive, PR-positive, and Her2-negative subsets of tumours. Finally, the association of MADE with other know prognostic variables including lymph node status, tumour size, grade and molecular subtypes, including PAM50 and IntClust, did not reveal any further significant association.

### Summary

The findings reveal the importance of assessing the expression levels of mutant oncogenic alleles as part of clinical management. In spite of the high frequency of PIK3CA mutation in breast cancers, the response to PI3K inhibitor therapy has not been as successful as expected, and the prognostic significance of detecting somatic PIK3CA mutations in breast tumour is unclear. Firstly, the examples here establish the prognostic relevance of transcriptional allelic regulation of somatic mutations in breast cancer, supporting a novel method for mutation testing in patient management, where the level of expression of mutations should be considered, and not only their detection at the DNA level. Secondly, there is a strong need to identify patients who will not respond to specific therapies to prevent unnecessary drug cytotoxicity without therapeutic benefits. The examples herein demonstrate not all PIK3CA somatic mutations, commonly considered driver events, are equally expressed in tumours, with some hardly expressing the mutations identified at the DNA level. If the targeted somatic mutation is not expressed, the inventors predict no benefit will come from the treatment mutation targeted treatment.

TABLE 1

| | Heterozygotes | | Preferentially | Mean | | | P t.test |
|---|---|---|---|---|---|---|---|
| daeSNP | with DAE * | | Expressed Allele | DAE * | SD | Min-Max | (one sample) |
| rs 7636454 | 24% | (5/21) | A | 0.68 | 0.04 | 0.63-0.76 | 5.70E−06 |
| rs3960984 | 57% | (12/21) | G | 1.11 | 0.46 | 0.64-2.28 | 5.91E−06 |
| rs3729679 | 20% | (7/35) | G | 0.85 | 0.21 | 0.61-1.17 | 6.07E−05 |
| | 3% | (1/35) | A | 0.63 | 0 | 0.63-0.63 | — |
| rs 12488074 | 14% | (3/21) | G | 0.74 | 0.09 | 0.61-0.82 | 8.06E−03 |
| rs4855093 | 9% | (2/22) | A | 0.74 | 0.04 | 0.70-0.78 | 3.23E−02 |
| | 5% | (1/22) | G | 1.1 | 0 | 1.10-1.10 | — |
| rs9838411 | 14% | (3/22) | A | 0.76 | 0.2 | 0.59-1.04 | 3.34E−02 |

* DAE calculated as log2(expression of allele 1/expression of allele 2); SD - Standard Deviation; P - p-value

TABLE 2

| Variable | N | METABRIC, N = 94¹ | | TCGA, N = 178¹ | |
|---|---|---|---|---|---|
| alpha | 272 | −0.96 | (−2.47, 5.75) | −0.76 | (−4.82, 7.78) |
| alpha_group | 272 | | | | |
| >wt | | 63 | (67%) | 96 | (54%) |
| neutral | | 23 | (24%) | 57 | (32%) |
| >mut | | 8 | (8.5%) | 25 | (14%) |
| gamma | 272 | 0.48 | (−1.22, 3.70) | 0.19 | (−2.10, 2.46) |
| gamma_group | 272 | | | | |
| >wt | | 6 | (6.4%) | 24 | (13%) |
| neutral | | 47 | (50%) | 96 | (54%) |
| >mut | | 41 | (44%) | 58 | (33%) |
| MADE | 272 | 47 | (50%) | 82 | (46%) |

¹Statistics presented: Median (Range); n (%)

TABLE 3

| QRSID | RSID | R2 | DPRIME |
|---|---|---|---|
| rs7636454 | rs4855093 | 0.91 | 0.97 |
| rs7636454 | rs3960984 | 0.90 | 0.98 |
| rs7636454 | rs9838411 | 0.91 | 0.98 |
| rs7636454 | rs3729679 | 0.22 | 1.00 |
| rs7636454 | rs12488074 | 0.99 | 1.00 |
| rs7636454 | rs7636454 | 1.00 | 1.00 |
| rs3960984 | rs4855093 | 0.98 | 1.00 |
| rs3960984 | rs3960984 | 1.00 | 1.00 |
| rs3960984 | rs9838411 | 0.99 | 1.00 |
| rs3960984 | rs3729679 | 0.20 | 0.99 |
| rs3960984 | rs12488074 | 0.89 | 0.98 |
| rs3960984 | rs7636454 | 0.90 | 0.98 |
| rs3729679 | rs4855093 | 0.20 | 0.98 |
| rs3729679 | rs3960984 | 0.20 | 0.99 |
| rs3729679 | rs9838411 | 0.21 | 0.99 |
| rs3729679 | rs3729679 | 1.00 | 1.00 |
| rs3729679 | rs12488074 | 0.22 | 1.00 |
| rs3729679 | rs7636454 | 0.22 | 1.00 |
| rs12488074 | rs4855093 | 0.90 | 0.97 |
| rs12488074 | rs3960984 | 0.89 | 0.98 |
| rs12488074 | rs9838411 | 0.91 | 0.98 |
| rs12488074 | rs3729679 | 0.22 | 1.00 |
| rs12488074 | rs12488074 | 1.00 | 1.00 |
| rs12488074 | rs7636454 | 0.99 | 1.00 |
| rs4855093 | rs4855093 | 1.00 | 1.00 |
| rs4855093 | rs3960984 | 0.98 | 1.00 |
| rs4855093 | rs9838411 | 0.99 | 1.00 |
| rs4855093 | rs3729679 | 0.20 | 0.98 |
| rs4855093 | rs12488074 | 0.90 | 0.97 |
| rs4855093 | rs7636454 | 0.91 | 0.97 |
| rs9838411 | rs4855093 | 0.99 | 1.00 |
| rs9838411 | rs3960984 | 0.99 | 1.00 |
| rs9838411 | rs9838411 | 1.00 | 1.00 |
| rs9838411 | rs3729679 | 0.21 | 0.99 |
| rs9838411 | rs12488074 | 0.91 | 0.98 |
| rs9838411 | rs7636454 | 0.91 | 0.98 |

TABLE 4

| daeSNP | SNP associated | Perm pvalue |
|---|---|---|
| rs3960984 | rs4854984 | 0.001 |
| rs3960984 | rs1468920 | 0.001 |
| rs3960984 | rs1468922 | 0.009 |
| rs3960984 | rs1468923 | 0.011 |
| rs3960984 | rs6804758 | 0.046 |
| rs3960984 | rs9822116 | 0.038 |
| rs3960984 | rs2111534 | 0.053 |
| rs4855093 | rs11719127 | 0.048 |
| rs4855093 | rs11920864 | 0.008 |
| rs4855093 | rs1976765 | 0.017 |
| rs4855093 | rs9814424 | 0.017 |
| rs4855093 | rs11706842 | 0.012 |
| rs4855093 | rs7641889 | 0.012 |
| rs4855093 | rs6786049 | 0.002 |
| rs4855093 | rs6799756 | 0.002 |
| rs4855093 | rs7645550 | 0.027 |
| rs3729679 | rs6443607 | 0.03 |
| rs3729679 | rs13092881 | 0.017 |
| rs3729679 | rs9879637 | 0.04 |
| rs3729679 | rs4501160 | 0.039 |
| rs3729679 | rs9841497 | 0.031 |
| rs3729679 | rs4583702 | 0.023 |
| rs3729679 | rs4955797 | 0.035 |
| rs3729679 | rs7652946 | 0.037 |
| rs3729679 | rs7612363 | 0.012 |
| rs3729679 | rs4579062 | 0.006 |
| rs3729679 | rs7639391 | 0.016 |
| rs3729679 | rs6790867 | 0.015 |
| rs3729679 | rs1542 | 0.017 |
| rs3729679 | rs7633318 | 0.013 |
| rs3729679 | rs6793893 | 0.016 |
| rs3729679 | rs6800015 | 0.028 |
| rs3729679 | rs4955807 | 0.021 |
| rs3729679 | rs6772028 | 0.032 |
| rs3729679 | rs6784495 | 0.044 |
| rs3729679 | rs2677770 | 0.046 |
| rs3729679 | rs12494623 | 0.001 |
| rs12488074 | rs9841497 | 0.015 |
| rs12488074 | rs4583702 | 0.017 |
| rs12488074 | rs4955797 | 0.014 |
| rs12488074 | rs6791364 | 0.019 |
| rs12488074 | rs7629064 | 0.036 |
| rs12488074 | rs6784495 | 0.018 |
| rs12488074 | rs9968179 | 0.022 |
| rs12488074 | rs2699887 | 0.027 |
| rs12488074 | rs2699905 | 0.23 |
| rs12488074 | rs2677770 | 0.017 |

TABLE 5

| | Overall Survival | | | | |
|---|---|---|---|---|---|
| Discovery set | N | Events | Median | 0.95LCL | 0.95UCL |
| MADE vs. no_MADE | | | | | |
| no_MADE | 46 | 21 | 6750 | 4138 | NA |
| MADE | 45 | 26 | 2907 | 2112 | NA |
| MADE groups | | | | | |
| MADE_low | 6 | 4 | 1382 | 1293 | NA |
| no_MADE | 46 | 21 | 6750 | 4138 | NA |
| MADE_high | 39 | 22 | 3617 | 2149 | NA |
| ER positive | | | | | |
| MADE_low | 5 | 3 | 2737 | 1293 | NA |
| no_MADE | 42 | 18 | 8750 | 4341 | NA |
| MADE_high | 28 | 12 | 4550 | 3617 | NA |
| ER neg | | | | | |
| MADE_low | 1 | 3 | 1382 | NA | NA |
| no_MADE | 4 | 3 | 2539 | 744 | NA |
| MADE_high | 11 | 10 | 985 | 939 | NA |
| PR pos | | | | | |
| MADE_low | 5 | 3 | 2737 | 1293 | NA |
| no_MADE | 37 | 14 | 6750 | 4680 | NA |
| MADE_high | 22 | 10 | 4550 | 3617 | NA |
| PR neg | | | | | |
| MADE_low | 1 | 3 | 1382 | NA | NA |
| no_MADE | 9 | 7 | 3660 | 1296 | NA |
| MADE_high | 17 | 12 | 1530 | 962 | NA |
| HER2 pos | | | | | |
| MADE_low | 0 | | | | |
| no_MADE | 6 | 3 | 4138 | 2048 | NA |
| MADE_high | 9 | 6 | 2149 | 1071 | NA |
| HER2 neg | | | | | |
| MADE_low | 6 | 4 | 1382 | 1293 | NA |
| no_MADE | 40 | 18 | 6750 | 4277 | NA |
| MADE_high | 30 | 16 | 3950 | 2907 | NA |
| | Disease Specific Survival | | | | |
| | N | Events | Median | 0.95LCL | 0.95UCL |
| MADE vs. no_MADE | | | | | |
| no MADE | 46 | 9 | NA | NA | NA |
| MADE | 45 | 18 | NA | 2582 | NA |
| MADE groups | | | | | |
| MADE_low | 6 | 3 | 1382 | 1293 | NA |
| no_MADE | 46 | 9 | NA | NA | NA |
| MADE_high | 39 | 15 | NA | 2907 | NA |
| ER positive | | | | | |
| MADE_low | 5 | 2 | NA | 1293 | NA |
| no_MADE | 42 | 8 | NA | NA | NA |
| MADE_high | 18 | 7 | NA | NA | NA |
| ER neg | | | | | |
| MADE_low | 1 | 1 | 1382 | NA | NA |
| no_MADE | 44 | 1 | NA | 744 | NA |
| MADE_high | 11 | 8 | 1374 | 939 | NA |
| PR pos | | | | | |
| MADE_low | 5 | 2 | NA | 1293 | NA |
| no_MADE | 37 | 7 | NA | NA | NA |
| MADE_high | 22 | 6 | NA | 3617 | NA |
| PR neg | | | | | |
| MADE_low | 5 | 2 | NA | 1293 | NA |
| no_MADE | 37 | 7 | NA | NA | NA |
| MADE_high | 22 | 6 | NA | 3617 | NA |
| HER2 pos | | | | | |
| MADE_low | 0 | 0 | | | |
| no_MADE | 6 | 2 | NA | 2048 | NA |
| MADE_high | 9 | 5 | 2149 | 1530 | NA |
| HER2 neg | | | | | |
| MADE_low | 6 | 3 | 1382 | 1293 | NA |
| no_MADE | 40 | 7 | NA | NA | NA |
| MADE_high | 30 | 10 | NA | 3447 | NA |

1. A method of predicting the prognosis of a cancer patient, particularly a breast cancer patient, diagnosed with a tumour characterised by a mutant PIK3CA allele
said method comprising:
a. determining in a tumour sample obtained from a patient:
i. the expression level of a mutant PIK3CA allele ( [symbol] ( [symbol] )), and
ii. the expression level of a wildtype PIK3CA allele ( [symbol] ( [symbol] ));
b. assigning the cancer patient
i. a likelihood of a good prognosis if: [symbol]
( [symbol] ) is about equivalent to [symbol] ( [symbol] ), or
ii. a likelihood of a poor prognosis if: [symbol]
( [symbol] ) is significantly greater than (>) [symbol] ( [symbol] ), or [symbol]
( [symbol] ) is significantly > [symbol] ( [symbol] ),
particularly wherein [symbol] ( [symbol] ) is significantly > [symbol] ( [symbol] );
wherein optionally, poor prognosis is defined as about a 45-55% or less probability of survival at about 11 years, and good prognosis is defined as about a 75-85% probability of survival at about 18 years.

2. The method according to claim 1, wherein the cancer patient is assigned a likelihood of poor prognosis if:
i. [symbol] ( [symbol] ) is at least 1.5 greater than (≥) [symbol] ( [symbol] ), or [symbol]
( [symbol] ) is ≥1.5 fold [symbol] ( [symbol] ),
particularly wherein [symbol] ( [symbol] ) is 1.5 fold ≥ [symbol] ( [symbol] ); or
ii. a likelihood of good prognosis if: [symbol]
( [symbol] ) is less than (<) 1.5 fold [symbol] ( [symbol] ), and [symbol]
( [symbol] ) is <1.5 fold [symbol] ( [symbol] ),

3. The method according to claim 1, wherein the cancer patient is assigned:
i. a likelihood of a very poor prognosis if [symbol]
( [symbol] ) is >1.5 fold [symbol] ( [symbol] ),
ii. a likelihood of poor prognosis if [symbol]
( [symbol] ) is >1.5 fold [symbol] ( [symbol] ), or
iii. a likelihood of good prognosis if [symbol]
( [symbol] ) is <1.5 fold [symbol] ( [symbol] ), and [symbol]
( [symbol] ) is <1.5 fold [symbol] ( [symbol] ),
wherein optionally poor prognosis is defined as about a 50-60% probability of survival at about 11 years, very poor prognosis is defined as about a 45-55% probability of survival at about 5 years, and good prognosis is defined as about a 75-85% probability of survival at about 18 years.

4. A method for treating a cancer patient, particularly a breast cancer patient said method comprising:
a. determining in a tumour sample obtained from the cancer patient:
i. the expression level of a mutant PIK3CA allele ( [symbol] ( [symbol] )),
ii. the expression level of a wildtype PIK3CA allele ( [symbol] ( [symbol] )),
b. evaluating whether: [symbol]
( [symbol] ) is>1.5 fold [symbol] ( [symbol] ), or
ii. [symbol] ( [symbol] ) is <1.5 fold [symbol] ( [symbol] ),
c. administering an effective dose of a PIK3 inhibitor drug to the cancer patient if [symbol] ( [symbol] ) is >1.5 fold [symbol] ( [symbol] ).

5. The method according to claim 1, wherein the expression level of the mutant PIK3CA allele and the expression level of wildtype PIK3CA allele are obtained using a quantitative, allele-specific, method to measure allele expression, particularly wherein the method is selected from mRNA sequencing, an mRNA array, or quantitative real time PCR;
more particularly wherein the PIK3CA expression level is obtained using mRNA sequencing, or an mRNA array, particularly an mRNA array.

6. The method according to claim 1, wherein the expression level of mutant PIK3CA allele is determined for at least one PIK3CA single nucleotide polymorphism (SNP) locus, particularly at least one PIK3CA SNP selected from:
a. rs3960984,
b. rs7636454,
c. rs3729679,
d. rs12488074,
e. rs2699887
f. rs4855093, and/or
g. rs9838411,
particularly wherein the expression level of mutant PIK3CA allele is determined for the SNP rs3960984.

7. The method according to claim 1, wherein the expression level of mutant PIK3CA allele is determined for a SNP located within an untranslated region of a mutant PIK3CA mRNA transcript, particularly wherein the expression level of mutant PIK3CA allele is determined for the SNP rs2699887.

8. The method according to claim 1, wherein the patient has been diagnosed with breast cancer.

9. The method according to claim 8, wherein the tumour that has been classified as:
a. oestrogen receptor positive, or negative,
b. progesterone receptor positive, or negative, and/or
c. human epidermal growth factor receptor 2 negative, or positive.

10. The method according to claim 4, wherein the PIK3 inhibitor drug is alpelisib or copanlisib, particularly wherein the PI3K inhibitor is alpelisib.

11. (canceled)

12. A kit for analysing biomarkers in order to determine a mutant PIK3CA allelic expression status of a tumour sample from a patient diagnosed with a cancer characterised by a PIK3CA mutation, particularly a breast cancer patient comprising primers for amplification and detection of expression of a mutant PIK3CA allele and a wildtype PIK3CA allele present at the locus of at least one SNP selected from rs7636454, rs2699887, rs3960984, rs3729679, rs12488074, rs4855093, and/or rs9838411, particularly rs3960984, or rs2699887,7.
